(19) United States
(12) Patent Application Publication
(10) Pub. No.: US 2002/0048752 A1
(43) Pub. Date: Apr. 25, 2002

(54) METHODS FOR DETECTING LOWER-FREQUENCY MOLECULES

(76) Inventors: STANLEY N. LAPIDUS, BEDFORD, NH (US); ANTHONY P. SHUBER, MILFORD, MA (US)

Correspondence Address: TESTA, HURWITZ & THIBEAULT, LLP, HIGH STREET TOWER, 125 HIGH STREET, BOSTON, MA, 02110, US

(21) Appl. No.: 09/441,760

(22) Filed: Nov. 17, 1999

Related U.S. Application Data
(60) Provisional application No. 60/109,724, filed on Nov. 23, 1998.

(51) Int. Cl. C12Q 1/68; C12P 19/34; C07H 21/02; C07H 21/04

(52) U.S. Cl. 435/6; 435/91.2; 536/23.1

(57) ABSTRACT

Methods are provided for detection of lower-frequency molecules in relation to and against the background of higher-frequency molecules.

### CROSS-REFERENCE TO RELATED APPLICATION

The present application claims priority to and the benefit of U.S. provisional patent application serial No. 60/109,724, filed Nov. 23, 1998, the entire disclosure of which is incorporated herein by reference.

### FIELD OF THE INVENTION

The present invention relates generally to methods for detecting lower-frequency molecules in biological samples.

### BACKGROUND OF THE INVENTION

In samples containing heterogeneous populations of nucleic acids, lower-frequency nucleic acids are difficult to detect with standard analysis methods. Often, the occurrence of the lower-frequency event is compared with a higher-frequency event (e.g., a mutant allele compared with a wild type allele). The two types of nucleic acids are visualized on a separation gel, side by side, in order to compare the relative amounts of each. Typically, radioactive, fluorescent, or other photo-emitting materials are used to label different types of nucleic acids. The result is a large signal (i.e., the higher-frequency event) in physical proximity to a much smaller signal (i.e., the lower-frequency event).

In many cases, a larger signal overwhelms a smaller signal. As a result, the smaller signal may not be distinct or detectable against the background of the larger signal (producing a false negative). Moreover, the smaller signal, if detectable, may be artificially large because “spillover” from the larger signal is detected and counted as the smaller signal (a type of false positive).

One incomplete solution to this problem is to physically separate the larger and smaller signals. For example, if signal is detected on a gel, the lanes containing the two nucleic acid molecules under analysis may be spaced apart. However, greater physical separation on, for example, a separation gel means fewer samples can be run simultaneously. Moreover, differences in gel concentration, electric field strength, and local heating may confound results. This solution increases cost and increases the time it takes to analyze samples. Another possible solution to the problem is provided by various advances in imaging technology that allow weaker signals to be detected. However, such technology cannot detect very low-frequency events relative to more frequent events and, also, does not completely eliminate “spillover.”False negative and false positive errors are common in any assay in which a relatively rare event is to be distinguished from a relatively more common event. When a lower-frequency event is measured relative to a higher-frequency event and is subject to background noise, the lower-frequency event may not be detected at all or the lower-frequency event may be detected when it is not present. The present invention overcomes these and other problems by providing methods for detecting lower-frequency molecular events.

### SUMMARY OF THE INVENTION

The present invention provides methods for detecting signal from a lower-frequency molecular event relative to and/or in a background of a higher-frequency molecular event. The invention provides solutions to both the problem of detecting a small signal against a background of a large signal, and the problem of spillover which causes an incorrect exaggeration of the small signal due to spillover by the large signal. According to methods of the invention, signal corresponding to a first molecule present in a sample in excess relative to a second (lower-frequency) molecule is reduced to approximate the signal corresponding to the lower-frequency molecule. Thus, if a lower-frequency molecule is present in the sample, its signal will not be obscured by signal from the more-prevalent species. The present invention is particularly useful when one wishes to compare the ratio of one molecule, present in excess in a sample, with another molecule, present with lower-frequency in a sample. Methods of the invention also provide significant cost savings. For example, methods of the invention require fewer separation gels to examine a nucleic acid sample for the presence of one or more nucleic acid species of interest. Moreover, less label is used for labeling the higher-frequency event than in standard techniques.

One embodiment of the invention is a method for identifying a low-frequency nucleic acid present in a heterogenous sample. In such methods, a first probe is capable of hybridizing with a portion of a higher-frequency nucleic acid in the sample, and a second probe is capable of hybridizing with a portion of the lower-frequency nucleic acid. Only a proportion of the first probe comprises a first detectable label. The proportion of labeled first probe to unlabeled first probe is approximately equal to a proportion of lower-frequency nucleic acid relative to higher-frequency nucleic acid. The remainder of the first probe is unlabeled. In contrast, the second probe comprises a second detectable label. Labeled and unlabeled first probe combined are nominally equimolar with the labeled second probe.

When first and second probes are exposed to a sample, the signal observed from the two different labels is approximately equivalent when the lower-frequency nucleic acid is present in the sample at the threshold proportion for detection (which is set relative to the assay and the level of confidence desired). Unlike the case in which the higher-frequency nucleic acid is labeled at or near saturation, when the proportion of the labeled first probe to unlabeled first probe is approximately equal to the proportion of the lower-frequency nucleic acid to higher-frequency nucleic acid, the signal from the higher-frequency nucleic acid will not overwhelm signal from the lower-frequency nucleic acid. Accordingly, the accuracy of measurement of the lower-frequency nucleic acid is increased. In preferred embodiments of the invention, labeled first and second probes comprise separate detectable labels. Preferred labels are selected from the group consisting of radioactive material, fluorescent material, light-emitting material, and electromagnetic radiation-emitting material.

In another embodiment of the invention, each of a first labeled oligonucleotide probe, a second labeled oligonucleotide probe, and a third unlabeled oligonucleotide probe are annealed to different portions of a nucleic acid in the sample. The first oligonucleotide probe anneals to a first portion of the nucleic acid in the sample; second oligonucleotide probe anneals to a second portion of the nucleic acid in the sample; and a third oligonucleotide probe anneals to at least part of the first portion of the nucleic acid in the sample. The first portion occurs more frequently than the second portion in the nucleic acid sample. The third oligonucleotide probe competes with the first oligonucleotide probe to bind to the first portion. Alternatively, the third probe prevents the first probe from binding the first portion. The nucleic acid sample is washed to remove unhybridized probe. The presence of the second signal determines that the less-frequent nucleic acid is present in the sample. Competition from the third probe ensures that signal from the first probe does not “spillover” to obscure true signal from the second probe.

In another preferred embodiment, methods of the invention are used in a single-base extension reaction to detect and/or identify a single nucleotide that is present in the sample in a lower-frequency amount relative to a higher-frequency nucleotide at that position (e.g., a single nucleotide polymorphic variant). In such methods, a primer is capable of hybridizing to a target nucleic acid at a locus on such target that is immediately 3′ to the single base to be detected. In the presence of a polymerase, the sample is exposed to at least two non-extendible nucleotides for incorporation into the extending primer. The non-extendible base that is expected to be complementary to the higher-frequency nucleotide in the sample at the position being interrogated includes a labeled first base and an unlabeled first base. The non-extendible base that is expected to be complementary to the lower-frequency nucleotide in the sample at the position being interrogated includes a labeled second base. The proportion of higher-frequency sites filled by labeled first base to that filled by unlabeled first base is approximately equal to the proportion of the lower-frequency nucleotide to the higher-frequency nucleotide. The proportions of the labeled first probe after extension and labeled second probe after extension are determined in order to reliably indicate the amount of the lower-frequency nucleotide without interference from signal attributable to the higher-frequency nucleotide. Unincorporated base can be washed away and the sample detected.

In further embodiments, methods of the invention described above using single-base extension may be accomplished with extendible 3′ nucleotides added to the extending primer. Moreover, detection may be accomplished in single base extension methods by attaching a donor molecule to the primer and attaching an acceptor molecule to the added nucleotides. When in close proximity (i.e., when a 3′ base comprising an acceptor molecule has been added), the donor molecule causes the acceptor to emit a signal that is characteristic of the donor-acceptor combination in close proximity (e.g., a characteristic wavelength of light associated with the donor/acceptor combination, but not with either one alone).

The invention will be understood further upon consideration of the following description and claims.

### DETAILED DESCRIPTION OF THE INVENTION

The invention provides methods for detection of lower-frequency nucleic acids in relation to and against the background of higher-frequency nucleic acids. When a lower-frequency nucleic acid (e.g., a mutant allele) exists in a biological sample, two problems arise when detecting the event relative to a higher-frequency event (e.g., a wild type allele). First, a signal attributable to the lower-frequency nucleic acid can be lost if a signal from the higher-frequency nucleic acid is strong. Second, a signal from the lower-frequency nucleic acid can be erroneously large if a signal from the higher-frequency nucleic acid “spills over” and is mistakenly detected as signal from the lower-frequency nucleic acid.

Labels or other materials that produce a signal include those materials that do not emit a signal by themselves but must be activated in order to emit a signal. Labeling a probe or a base with a signal includes labeling before hybridization, during hybridization, or after hybridization. For the sake of simplicity, the embodiments of the invention described herein only disclose methods involving one lower-frequency nucleic acid or nucleotide. However, the invention provides for methods that are equally applicable to situations in which more than one lower-frequency nucleic acid or nucleotide is to be detected (e.g., multiplexing assays). Oligonucleotide probe (or simply “probe”) or oligonucleotide primer (or simply “primer”) is meant to refer to any nucleic acid or analog thereof, including protein nucleic acids, capable of Watson-Crick type base pairing.

I. Probe-Based Methods of the Invention

Methods of the invention are used to detect a small proportion of mutant DNA present in a stool sample at the early stages of colorectal cancer development by analysis of the ratio of wild-type to mutant DNA in the sample. A stool sample is obtained from a patient. Approximately 2 grams of a representative stool sample is obtained according to the teachings of U.S. Pat. No. 5,741,650, incorporated by reference herein. The stool sample is homogenized in 40 ml physiologically-compatible buffer at a buffer: stool ratio of about 20:1. After homogenization, DNA is isolated from the sample by known methods. In an alternative embodiment, DNA is pooled from a plurality of patient samples.

The DNA sample is then exposed to labeled and unlabeled first probe and labeled second probe. The labeled second probe is complementary with a portion of the DNA expected to contain a mutation in patients with colorectal cancer. The labeled and unlabeled first probe is complementary with a portion of the DNA in the sample known not to be mutated in colorectal cancer. Because the mutant to be detected is assumed to be present in the sample as approximately 1% of the total DNA, approximately 1% of the first probe comprises a first detectable label, and 99% of the first probe is unlabeled. Labeled and unlabeled first probe combined are nominally equimolar with the labeled second probe, and unlabeled first probe can be in a molar amount in excess of labeled first probe. Upon exposure to the sample, the labeled second probe approximates saturation at the mutant DNA hybridization site (i.e., the second portion). The labeled and unlabeled first probe compete for hybridization with wild-type DNA. Preferably, the amount of bound labeled first probe is approximately equal to the amount of bound labeled second probe. An alternative embodiment includes the steps described above, but the concentrations of labeled first probe and labeled second probe can be substantially equal.

After hybridization is complete, the sample is washed to remove any unbound probe. Bound probe is melted from target, and the amounts of the first labeled probe and labeled second probes are detected. In this example, ³³P and ³²P radiolabels are used for first label and second label, respectively. However, calorimetric, mass, or other markers also work well. If mutant DNA is present in the sample, the proportions of signal from the first probe and signal from the second probe are approximately equal. Because the proportions are approximately equal, resolution of the signal from each (and particularly from the mutant-associated signal) is improved over the situation in which all the wild-type DNA is labeled (i.e., only labeled first probe and no unlabeled first probe is added). The mutant signal is detectably distinct from the wild type signal. For example, there is no spillover from the excess (wild-type) label. This allows superior measurement of the ratio of wild-type to mutant in order to determine whether mutant levels exceed the statistical criteria of the assay (i. e., whether it can be said that the mutant exists in the sample at or above the threshold for determination that a mutant subpopulation of cancerous or precancerous cells exists in the sample). In one embodiment of the invention, the intensity difference between a first signal and a second signal is less than two orders of magnitude. If the mutant DNA is determined to exist in the sample, the patient is advised that he or she should seek confirmation through subsequent testing.

Parameters of methods according to the inventor are varied depending on the assay system employed. For example, annealing conditions may be varied. The melting temperature (Tm) of the hybridization determines binding. One calculates Tm, for example, according to the formula Tm (° C.)=2(number of A+T residues)+4(number of G+C residues). The Tm also depends on the type of nucleic acid comprising the probe/target pair. For example, the Tm of RNA/RNA>RNA/DNA>DNA/DNA. Other reaction conditions effect hybridization, such as salt concentration.

Additionally, multiple labeling methods are appropriate for use with the methods of the invention. For example, labeling methods utilizing radioactive labels, fluorescent labels, light-emitting labels, or other electromagnetic radiation-emitting labels are adaptable according to the methods of the invention. Labeled probes preferably comprise a “signal moiety” which facilitates detection of the probes that have been hybridized to a nucleic acid sample. Signal moieties can be fluorescent, luminescent or radioactive labels, enzymes, haptens, and other chemical tags such as biotin which allow for easy detection of labeled extension products. Fluorescent labels such as the dansyl group, fluorescein and substituted fluorescein derivatives, acridine derivatives, coumarin derivatives, pthalocyanines, tetramethylrhodamine, Texas Red®, 9-(carboxyethyl)-3-hydroxy-6-oxo-6H-xanthenes, DABCYL® and BODIPY® (Molecular Probes, Eugene, Ore.), for example, are suitable for the methods described herein. Such labels are routinely used with automated instrumentation for simultaneous high throughput analysis of multiple samples.

II. Primer-Based Methods of the Invention

Methods of the invention are used to detect a small percentage of mutant DNA present in a stool sample at the early stages of colorectal cancer development by analysis of the ratio of wild-type to mutant DNA in the sample. A stool sample is obtained from a patient. Approximately 2 grams of a representative stool sample is obtained according to the teachings of U.S. Pat. No. 5,741,650, incorporated by reference herein. The stool sample is homogenized in 40 ml buffer at a buffer:stool ratio of about 20:1. After homogenization, DNA is isolated from the sample by known methods. In a preferred embodiment, the DNA is amplified by, for example, PCR.

Sample is exposed to a first primer that binds a first portion of the nucleic acid sample that lies 3′ to the nucleotide to be interrogated. This nucleotide is either a higher-frequency nucleotide (i.e., the wild type allele) or a lower-frequency nucleotide (i.e., the mutant allele).

The first primer is extended, for example, by Polymerase in the presence of a labeled first base complementary to the higher-frequency nucleotide, an unlabeled first base complementary to the higher-frequency nucleotide, and a labeled second base complementary to the lower-frequency nucleotide. The labeled second base is complementary with the mutant base (e.g., the patient from whom the sample is obtained is in the early stages of colorectal cancer). The labeled and unlabeled first base is complementary with a portion of the DNA in the sample known not to be mutated in colorectal cancer. Because the mutant to be detected is present in the sample at approximately 1% of the total DNA, 1% of the first base comprises a labeled first base, and 99% of the first base comprises an unlabeled first base. Labeled and unlabeled first base combined are nominally equimolar with labeled second base, and unlabeled first base can be in a molar amount in excess of labeled first base. Unincorporated base can be washed away prior to detecting the components of the sample. For convenience, the sample may be divided into first and second aliquots for separate analysis of first and second nucleotide incorporation. An alternative embodiment includes the steps described above, but the concentrations of labeled first base and labeled second base can be substantially equal.

In one embodiment of the invention, the intensity difference between a first signal and a second signal is less than two orders of magnitude. If the mutant DNA is determined to exist in the sample, the patient is advised that he or she should seek confirmation through subsequent testing.

Any nucleotide chain amplification method which incorporates an unlabeled base or labeled base is useful according to the methods of the present invention. For example, PCR protocols, transcription protocols, ligase chain reaction, ARMS, and reverse transcription protocols all incorporate nucleotides into a growing chain in a primer-dependent manner. Moreover, in one embodiment, a labeled first base, an unlabeled first base, and/or labeled second base are a nucleotide capable of terminating the growth of a nucleotide chain once incorporated (e.g., a dideoxynucleotide).

Additionally, multiple labeling methods are appropriate for use with the methods of the invention. For example, labeling methods utilizing radioactive labels, fluorescent labels, light-emitting labels, or other electromagnetic radiation-emitting labels are adaptable according to the methods of the invention.

Oligonucleotide primers of the present invention include segmented primers. One embodiment of the methods of the invention comprises using segmented primers to enhance template-dependent nucleic acid polymerization. Such methods are especially useful for detection of mutations, especially point mutations. Methods of the embodiment of the invention comprise hybridizing two probes adjacent to a site of suspected mutation, wherein neither probe alone is capable of being a primer for template-dependent extension, but wherein adjacent probes are capable of priming extension (i. e., a segmented primer). In a preferred embodiment, methods of the invention comprise hybridizing to a target nucleic acid a probe having a length from about 5 bases to about 10 bases, wherein the probe hybridizes immediately upstream of a suspected mutation. Methods of the invention further comprise hybridizing a second probe upstream of the first probe, the second probe having a length from about 15 to about 100 nucleotides and having a 3′ non-extendible nucleotide. The second probe is substantially contiguous with the first probe. Preferably, substantially contiguous probes are between 0 and about 1 nucleotide apart. A linker is preferably used where the first and second probes are separated by two or more nucleotides, provided the linker does not interfere with the nucleic acid extension reaction. Such linkers are known in the art and include, for example, peptide nucleic acids, DNA binding proteins, and ligation. Finally, methods of the embodiment of the invention comprise conducting an extension reaction to add a nucleotide to the segmented primer, and to detect it. In a preferred embodiment, a labeled first base, an unlabeled first base (or chain-terminating base), and a labeled second base (or chain-terminating base) are specific for the higher-frequency nucleotide in the case of the labeled and unlabeled first bases (e.g., a wild type species) or the lower-frequency nucleotide in the case of the labeled second base (e.g., a mutant or point mutation mutant). The unlabeled first base effectively dilutes the labeled first base, such that a first signal and a second signal produced by the labeled first base and the labeled second base are detectably distinct. These three bases, for example, are chain-terminating dideoxynucleotides.

Labeled ddNTPs or dNTPs preferably comprise a “signal moiety” which facilitates detection of the primers that have been extended with a labeled nucleotide. Signal moieties can be fluorescent, luminescent or radioactive labels, enzymes, haptens, and other chemical tags such as biotin which allow for easy detection of labeled extension products. Fluorescent labels such as the dansyl group, fluorescein and substituted fluorescein derivatives, acridine derivatives, coumarin derivatives, pthalocyanines, tetramethylrhodamine, Texas Red®, 9-(carboxyethyl)-3-hydroxy-6-oxo-6H-xanthenes, DABCYL® and BODIPY® (Molecular Probes, Eugene, Ore.), for example, are suitable for the methods described herein. Such labels are routinely used with automated instrumentation for simultaneous high throughput analysis of multiple samples.

What is claimed is:

1. A method for identifying a low-frequency nucleic acid in a biological sample, the method comprising the steps of:
(A) annealing a labeled first oligonucleotide probe to a first, higher-frequency nucleic acid in a biological sample under conditions that promote complementary hybridization between said labeled first oligonucleotide probe and at least a portion of said first, higher-frequency nucleic acid;
(B) annealing a labeled second oligonucleotide probe to a second, lower-frequency nucleic acid under conditions that promote complementary hybridization between said labeled second oligonucleotide probe and at least a portion of said second, lower-frequency nucleic acid; and
(C) annealing an unlabeled first oligonucleotide probe to said first, higher-frequency nucleic acid under conditions that promote complementary hybridization between said unlabeled first oligonucleotide probe and said portion of said first, higher-frequency nucleic acid, whereby said unlabeled first oligonucleotide probe competes with said labeled first oligonucleotide probe for binding at said portion of said first, higher-frequency nucleic acid such that a second signal from said labeled second oligonucleotide probe is detectably distinct from a first signal from said labeled first oligonucleotide probe.

2. The method of claim 1 wherein said labeled first oligonucleotide probe and said unlabeled first oligonucleotide probe combined comprise an equimolar amount with said labeled second probe.

3. The method of claim 1 wherein a concentration of said labeled first oligonucleotide probe and a concentration of said labeled second oligonucleotide probe are substantially equal.

4. The method of claim 1 wherein said unlabeled first oligonucleotide probe is present in a molar amount in excess of said labeled first oligonucleotide probe.

5. The method of claim 1 wherein detectable amounts of said first signal are substantially equal to detectable amounts of said second signal.

6. The method of claim 1 wherein each of said first signal and said second signal comprise an indication arising from a substance selected from the group consisting of radioactive material, fluorescent material, light-emitting material, and electromagnetic radiation-emitting material.

7. The method of claim 1 further comprising the steps of:
(D) washing said sample to remove unhybridized probe; and
(E) detecting a second signal from said labeled second oligonucleotide probe, said second signal being detectably distinct from a first signal from said labeled first oligonucleotide probe.

8. A method for identifying a low-frequency nucleic acid in a biological sample, the method comprising the steps of:
(A) annealing at least a first oligonucleotide primer to a nucleic acid in a biological sample under conditions that promote complementary hybridization between said first oligonucleotide primer and at least a portion of said nucleic acid;
(B) extending said annealed first oligonucleotide primer by at least one base, whereby said extension occurs in the presence of a labeled first base, an unlabeled first base, and a labeled second base; and
(C) detecting a second signal from said labeled second base, said second signal being detectably distinct from a first signal from said labeled first base.

9. The method of claim 8 wherein each of said labeled first base, said unlabeled first base, and said labeled second base are chain-terminating.

10. The method of claim 8 wherein said labeled first base and said unlabeled first base combined are present in approximately equimolar amounts with said labeled second base.

11. The method of claim 8 wherein said unlabeled first base comprises a molar amount in excess of said labeled first base.

12. The method of claim 8 wherein a concentration of said labeled first base and a concentration of said labeled second base are substantially equal.

13. The method of claim 8 wherein detectable amounts of said first signal are substantially equal to detectable amounts of said second signal.

14. The method of claim 8 wherein each of said first signal and said second signal comprise an indication arising from a substance selected from the group consisting of radioactive material, fluorescent material, light-emitting material, electromagnetic radiation- emitting material.

15. The method of claim 8 further comprising the step of repeating steps (A) and (B).

16. The method of claim 8 wherein said first oligonucleotide primer is a segmented primer.

17. The method of claim 8 further comprising the step of washing away unincorporated base.
